(19) United States
(12) Patent Application Publication
(10) Pub. No.: US 2024/0117419 A1
(43) Pub. Date: Apr. 11, 2024

(54) CARBON COATED MAGNETIC PARTICLES AND METHODS FOR MAKING AND USING

(71) Applicant: University of Idaho, Moscow, ID (US)

(72) Inventors: I. Francis Cheng, Moscow, ID (US); Dipak Koirala, Moscow, ID (US); Peter B. Allen, Everett, WA (US); Forrest Dalbec, Superior, WI (US); Jeremy May, Tacoma, WA (US); Kailash Hamal, Moscow, ID (US); Nolan Nicholas, Moscow, ID (US)

(21) Appl. No.: 18/473,769

(22) Filed: Sep. 25, 2023

Related U.S. Application Data
(60) Provisional application No. 63/410,931, filed on Sep. 28, 2022.

(51) Int. Cl. C12Q 1/6855; B22F 1/05; B22F 1/102; B22F 1/16; C12Q 1/689

(52) U.S. Cl. CPC C12Q 1/6855; B22F 1/05; B22F 1/102; B22F 1/16; C12Q 1/689; B22F 2301/35; B22F 2302/40; B22F 2304/10

(57) ABSTRACT

Disclosed herein is a particle comprising a magnetic material and a carbon layer on the outer surface of the magnetic material. The carbon layer may comprise graphite, such as Graphite from the University of Idaho Thermolyzed Asphalt Reaction (GUITAR). The particle may further comprise a single stranded nucleic acid moiety, such as single stranded DNA or single stranded RNA, conjugated to the carbon layer. The disclosed particles are useful as nucleic acid sensors, PCR reagents, and in nucleic acid synthesis applications.

### CROSS REFERENCE TO RELATED APPLICATION

This application claims the benefit of the earlier filing date of U.S. provisional patent application No. 63/410,931, filed Sep. 28, 2022, which is incorporated herein by reference in its entirety.

### SEQUENCE LISTING

The nucleic and amino acid sequences listed in the accompanying sequence listing are shown using standard letter abbreviations for nucleotide bases, and single letter code for amino acids, as defined in 37 C.F.R. 1.822. Only one strand of each nucleic acid sequence is shown, but the complementary strand is understood as included by any reference to the displayed strand. The Sequence Listing is submitted as an XML file in the form of the file named “7832-109109-02_ST26.xml” (˜15,087 bytes), which was created on Sep. 21, 2023 which is incorporated by reference herein.

In the accompanying sequence listing:
SEQ ID NO. 1 is an exemplary single stranded DNA for attachment to the disclosed magnetic particles.
SEQ ID NO. 2 is a single stranded DNA that is complementary to SEQ ID NO. 1.
SEQ ID NO. 3 is a single stranded DNA that contains 4 mis-matched bases compared to SEQ ID NO. 2.
SEQ ID NO. 4 is a target DNA sequence for PCR.
SEQ ID NO. 5 is an amine modified DNA primer for PCR.
SEQ ID NO. 6 is a DNA primer or PCR.
SEQ ID NO. 7 is a DNA sequence generated by extension of a primer in a PCR process. target.
SEQ ID NO. 8 is a DNA moiety added to a primer by PCR to form a copy of the
SEQ ID NO. 9 is a target DNA sequence for PCR.
SEQ ID NO. 10 is an amine modified DNA primer for PCR.
SEQ ID NO. 11 is a DNA primer or PCR.
SEQ ID NOS. 12-14 are random DNA sequences used for comparison.

### FIELD

The application concerns carbon-coated magnetic particles that may further comprise a nucleic acid moiety and methods for making and using the particles.

### BACKGROUND

There is an urgent need for detection of specific nucleotide sequences for pathogens, and forensic analyses. Biomolecules can be attached to a particle surface by a variety of strategies, none of which are satisfactory for PCR. Non-covalent interactions include physisorption, Au—thiol bonds, and affinity binding. None of these couplings are stable under the conditions required for PCR. Covalent bonds are more stable. However, typical substrates for covalent bonding, such as polystyrene or latex, are not stable for the purposes of PCR amplification. Polymers with embedded Fe₃O₄ can be magnetically separated but suffer the same problems of intolerance to heat and low magnetic susceptibility.

### SUMMARY

Disclosed herein are aspects of a composition comprising a magnetic material particle that has an outer surface, and a carbon layer located on the outer surface of the magnetic material particle. In some aspects, the magnetic material particle is an iron particle. In some aspects, the carbon layer covers greater than 95% of the surface of the magnetic material particle, and may cover 99% or more of the surface of the magnetic material particle. In some aspects, the carbon layer comprises, or is, graphite, and may be graphite from the University of Idaho thermolyzed asphalt reaction (GUITAR).

The magnetic material particle may have an average particle size of from 5 μm to 100 μm, such as from 15 μm to 40 μm. And/or the magnetic material particle, together with the carbon layer, may have an average particle size of from 15 μm to 40 μm.

In any aspects, the carbon layer may be functionalized to facilitate conjugation to a desired moiety, such as a biomolecule, catalyst, or a combination of. In some aspects, the carbon layer is functionalized with 4-amino benzoic acid.

In some aspects, the composition further comprises a single stranded nucleic acid. The nucleic acid may have a length of from 10 bases to 1,000 bases or more, such as from 20 bases to 50 bases, or from 200 bases to 500 bases. The single stranded nucleic acid may be single stranded DNA, and in some aspects, the single stranded DNA is a PCR primer. In other aspects, the single stranded DNA is complementary to a target or analyte DNA moiety.

In certain aspects, the composition is a particulate composition where each particle of the particulate composition comprises an iron particle substantially coated with a carbon layer, where the carbon layer comprising graphite from the University of Idaho thermolyzed asphalt reaction (GUITAR). Additionally, the particulate composition comprises single stranded DNA conjugated to the carbon layer through an amino benzoic acid moiety on the carbon layer, the single stranded DNA having a length of from 20 bases to 500 bases. And each particle of the particulate composition has an average particle size of from 1 μm to 30 μm.

Also disclosed herein are aspects of a method comprising forming a mixture comprising a magnetic particle and a single stranded nucleic acid molecule in a solution, adding an intercalating agent to the mixture, separating the magnetic particle from the solution, and measuring the UV absorbance of the intercalating agent in the solution. The magnetic particle comprise a magnetic material particle comprising an outer surface, a carbon layer located on the outer surface of the magnetic material particle, and a single stranded nucleic acid moiety conjugated to the carbon layer, where the single stranded nucleic acid moiety is complementary to the single stranded nucleic acid molecule.

Further disclosed here are aspects of a method, comprising forming a mixture comprising a magnetic particle and a first PCR primer moiety, and a target nucleic acid strand, and performing a PCR process using the mixture. The magnetic particle comprises a magnetic material particle comprising an outer surface, a carbon layer located on the outer surface of the magnetic material particle, and a second PCR primer moiety.

Disclosed herein are aspects of a method for making a plurality of particles disclosed herein. The method comprises exposing magnetic material particles and silica particles to a first CVD process in the presence of a first CVD precursor, separating particles of a desired size from agglomerated particles, exposing the particles of a desired size to a second CVD process in the presence of a second CVD precursor to form a carbon layer on the magnetic material particles, and separating magnetic particles that are coated with the carbon layer from the silica particles to form the plurality of the disclosed particles. The method may further comprise functionalizing the carbon layer on the plurality of particles by treating the particles with 4-amino benzoic acid in the presence of an inorganic base, and conjugating the plurality of particles to a single stranded nucleic acid moiety.

The foregoing and other objects, features, and advantages of the disclosure will become more apparent from the following detailed description, which proceeds with reference to the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram illustrating an exemplary embodiment of a method for functionalizing the disclosed coated magnetic particles.

FIG. 2 is a schematic diagram illustrating a CVD reactor with a manual rotary kiln.

FIG. 3 is a scanning electron microscopy image illustrating the iron particles before coating with GUITAR.

FIG. 4 is a scanning electron microscopy image illustrating the iron particles after coating with GUITAR.

FIG. 5 is a scanning electron microscopy image illustrating benzoic acid functionalized GUITAR coated iron particles.

FIG. 6 is a schematic diagram illustrating an exemplary embodiment of a method for conjugating single stranded DNA (ss-DNA) onto the disclosed magnetic particles.

FIG. 7 is a schematic diagram illustrating the Beer's Law absorption signal produced by an ethidium bromide solution containing a disclosed embodiment of the ss-DNA modified magnetic particles.

FIG. 8 is a schematic diagram illustrating coupling the complementary DNA strand to the ss-DNA particle.

FIG. 9 provides the double-stranded DNA (ds-DNA) structure from one exemplary embodiment of the disclosed technology.

FIG. 10 is a schematic diagram illustrating obtaining the Beer's Law absorption signal after coupling the complementary DNA strand.

FIG. 11 is a graph of absorbance versus wavelength, illustrating the Beer's Law absorbance signal for ethidium bromide.

FIG. 12 is a graph of absorbance versus ethidium bromide concentration, illustrating linear relationship between concentration and absorbance.

FIG. 13 is a graph of absorbance versus composition, illustrating the absorbance of ethidium bromide in different combinations of the disclosed particles and single- or double-stranded DNA.

FIG. 14 provides an exemplary mis-matched double-stranded DNA structure.

FIG. 15 is a graph of absorbance versus wavelength, illustrating the decrease in absorbance of ethidium bromide due to intercalation into ds-DNA.

FIG. 16 is a graph of optical absorbance versus concentration illustrating the increasing absorbance loss with increasing concentration of the target ssDNA.

FIG. 17 is a Log absorbance plot as a function of concentration illustrating the linear relationship between signal and analyte ssDNA.

FIG. 18 is a graph of absorbance versus wavelength, illustrating the Beer's Law absorbance signal for crystal violet.

FIG. 19 is a graph of absorbance versus crystal violet concentration, illustrating linear relationship between concentration and absorbance.

FIG. 20 is a graph of absorbance versus composition, illustrating the absorbance of crystal violet in different combinations of the disclosed particles and single- or double-stranded DNA.

FIG. 21 is a schematic diagram illustrating one exemplary method of using the disclosed particles with the Polymerase Chain Reaction (PCR) process.

FIG. 22 is an agarose gel electrophoresis under UV 254 nm illumination of products from an exemplary PCR process at different cycles.

FIG. 23 is a schematic flowchart illustrating steps 1-4 of an exemplary PCR methodology using the disclosed magnetic particles.

FIG. 24 is a schematic flowchart illustrating steps 5-8 of the exemplary PCR methodology from FIG. 23 using the disclosed magnetic particles.

FIG. 25 is a schematic diagram illustrating the PCR process from Example 10.

FIG. 26 is a graph of absorbance versus ssDNA concentration, illustrating the absorbance at different target ssDNA concentrations with and without random sequences being present.

### DETAILED DESCRIPTION

### I. DEFINITIONS AND TERMS

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference in their entireties.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is recited.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

5AmMC6: Refers to the moiety

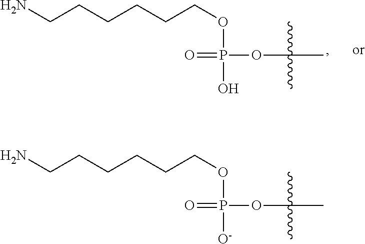 [SMILES: *OP(=O)(O)OCCCCCCN ; *OP(=O)([O-])OCCCCCCN]

in an unprotonated state. It is often used to provide an amine functional group to the 5′ end of an oligonucleotide.

cDNA: copy DNA (also known as complementary DNA) is DNA that has been transcribed from a specific mRNA in a reaction using the enzyme reverse transcriptase. Typically, cDNA contains only coding sequences.

Nucleic acid sequence: Refers to DNA and RNA sequences, such as cDNA and mRNA. In one examples, includes antisense nucleic acid sequences (such as antisense RNA or antisense DNA), microRNAs (miRNAs), small interfering RNAs (siRNAs), and repeat-associated small interfering RNAs (rasiRNAs). In one example, a nucleic acid sequence is a therapeutic nucleic acid sequence, such as a DNA therapeutic (e.g., antisense oligonucleotide, DNA aptamers) or RNA therapeutic (e.g., miRNA, siRNA, ribozyme, or RNA decoy). A nucleic acid sequence can include naturally occurring and/or non-naturally occurring nucleotides.

Nucleosides: The major nucleosides of DNA are deoxyadenosine (dA), deoxyguanosine (dG), deoxycytidine (dC) and deoxythymidine (T). The major nucleosides of RNA are adenosine (rA), guanosine (rG), cytidine (rC) and uridine (U). Includes nucleosides containing modified bases and modified sugar moieties, for example as described in U.S. Pat. No. 5,866,336 to Nazarenko et al. (herein incorporated by reference). Examples of modified sugar moieties which may be used to modify nucleotides at any position on its structure include, but are not limited to: arabinose, 2-fluoroarabinose, xylose, and hexose. In one example, a nucleoside is a 2′-deoxynucleoside (dA, dC, dG, or T). In one example, a nucleoside is chemically modified (e.g., LNA, BNA or UNA).

Peptide: A compound comprising amino acid residues connected by peptide bonds. As used herein, a peptide compound has from 2 to 7 or more amino acid residues.

Polypeptide: A compound comprising amino acid residues connected by peptide bonds. When the amino acids are alpha-amino acids, either the L-optical isomer or the D-optical isomer can be used. In some embodiments, a polypeptide has from about 50 amino acid residues to 2000 or more amino acid residues.

Protein: A molecule or complex comprising one or more polypeptides having secondary, tertiary and/or quaternary structure. The secondary, tertiary and/or quaternary structure of a protein typically is stabilized using non-covalent bonds, such as ionic bonds, hydrogen bonds, hydrophobic interactions, and/or van der Walls interactions. Additionally, or alternatively, a protein may include disulfide bonds, such as between the thiol groups of cysteine residues. In certain embodiments, a peptide, polypeptide, or protein comprises (e.g., consists essentially of) between 3 and 10, between 10 and 30, between 30 and 100, between 100 and 300, between 300 and 1,000, between 1,000 and 3,000, or between 3,000 and 10,000, inclusive, amino acids. In certain embodiments, the amino acids are natural amino acids. In certain embodiments, the amino acids are unnatural amino acids. In certain embodiments, the amino acids are L-amino acids. In certain embodiments, the amino acids are D-amino acids.

### II. OVERVIEW

There are several features useful for detection of specific biomolecules, such as nucleotide sequences, enzymes, antibodies, or proteins, for applications such as pathogen detection, genetic analysis, and forensic analyses. The features include (I) fast separation of isolated biomolecules from the sample and/or reaction matrix, and (II) low limits of detection which, in the case of nucleic acid sequences, may require the ability to conduct the polymerase chain reaction (PCR) on the isolate. For the possibility of feature (I), surface-immobilized DNA or RNA on magnetic and/or fluorescent microparticles is a useful option. It can be used for multiplex detection, sample preparation, target enrichment, single strand DNA or RNA generation. For feature (II), an advantageous property is creating a thermostable (for example, up to 100° C.) bond of the nucleotide to particle substrate that is stable under PCR conditions.

Disclosed herein is strategy that addresses the shortcomings described above. For feature (I), analyte oligonucleotide capture was made possible with magnetically susceptible microparticles, which offered rapid separation.

For feature (II) a robust covalent bond was formed with a complementary nucleic acid strand, such as a DNA strand, that was able to withstand conditions for PCR. These goals were accomplished by chemical vapor deposition of a pseudo-graphitic carbon onto iron microparticles (μ-Fe). The carbon is Graphite from the University of Idaho Thermolyzed Asphalt Reaction (GUITAR), which is rich in planar and interplanar structural defects. Additional information concerning the University of Idaho Thermolyzed Asphalt Reaction (GUITAR) can be found in U.S. Pat. Nos. 9,691,556 and 10,804,041, U.S. patent application Ser. No. 13/366,022, and international patent application No. PCT/US2010/044269, all of which are incorporated herein by reference in the entireties.

In an exemplary embodiment, these defects allowed covalent bond formation by functionalizing the material's surface with carboxyl groups followed by 5′ amine-modified DNA coupling with N-Ethyl-N′-(3-dimethylaminopropyl) carbodiimide hydrochloride (EDC). The analyte DNA sequence was allowed to hybridize to the immobilized complementary strand. A ds-DNA intercalator, ethidium bromide, was introduced. Hybridization detection was actuated by a reduction of an optical absorbance signal at 285 nm when a magnet separated the μ-Fe-bound dsDNA from the sample matrix. Similar results were obtained with other DNA intercalators, such as crystal violet (CV). The immobilized ssDNA was able to withstand the conditions required for PCR amplification. To the inventors' best knowledge, this is the first instance of nucleotide-modified magnetic carbon PCR-compatible substrate. In an example study, this technique exhibited a 5 pM limit of detection (LOD) with a 48 base pair sequence of Salmonella typhimurium.

Additionally, the disclosed particles and associated pathways do not require expensive equipment, for example, fluorescence or time-consuming electrophoretic separations. And unlike other immobilized-DNA or RNA systems, the disclosed magnetic particles are heat-stable and PCR compatible.

### III. COMPOSITION

Disclosed herein is a composition useful as a biosensor for biomolecule detection, such as detection of DNA, RNA, proteins, enzymes and/or antibodies. Additionally, or alternatively, the composition may be useful for catalysis, for example, where a catalyst can be easily removed from a mixture. In some aspects, the composition comprises a magnetic core at least partially coated with carbon, such as graphite. The carbon layer is functionalized with a functional group suitable for coupling to a biomolecule, for example, a nucleic acid moiety, such as a DNA moiety, RNA moiety, or a combination thereof.

In some aspects, the magnetic material comprises iron particles, such as iron microparticles (μ-Fe). The magnetic material, such as iron microparticles, may have an average size suitable for use in the disclosed technology. The average size may be from one nanometer or less to 10 millimeters or more, such as from 10 nanometers to 1 millimeter, or from 100 nanometers to 0.5 millimeters. In some aspects, the magnetic material has an average size of from 1 μm to 100 μm, such as from 5 μm to 100 μm, 5 μm to 75 μm, from 5 μm to 50 μm, from 10 μm to 50 μm, from 15 μm to 40 μm, from 20 μm to 35 μm, or from 25 μm to 30 μm. The average size of the magnetic material is determined by SEM, and refers to the longest dimension of the particle.

A carbon layer is located on the surface of the magnetic material. The carbon layer may partially or fully cover the surface of the magnetic material. In some aspects, the carbon layer covers at least 50% of the surface of the magnetic material, such as at least 60%, at least 70%, at least 80%, at least 90%, at least 95%, at last 98%, or at least 99% of the surface of the magnetic material. In certain aspects, the composition comprises an iron microparticle and a carbon layer on the surface of the microparticle, the layer covering at least 90% of the microparticle, such as at least 95%, at least 98%, or at least 99%. In some aspects, substantially all (e.g., at least 99%, such as at least 99.5%, or at least 99.9%) of the surface of the microparticle is covered by the carbon layer. Without being bound to a particular theory, substantially complete coverage of the magnetic particle, such as iron particle, prevents nucleic acid material from adsorbing onto the magnetic material, and also prevents sample contamination by the magnetic material.

In some aspects, the composition is a particulate composition comprising particles having an average size suitable for use in the disclosed technology. The particles may have an average size of from 1 μm to 10 millimeters or more, such as from 1 μm to 1 millimeter, or from 1 μm to 0.5 millimeters. In some aspects, the particles have an average size of from 1 μm to 250 μm, such as from 1 μm to 200 μm, from 1 μm to 150 μm, from 1 μm to 100 μm, from 5 μm to 100 μm, from 5 μm to 75 μm, from 5 μm to 50 μm, from 10 μm to 50 μm, from 15 μm to 40 μm, from 20 μm to 35 μm, or from 25 μm to 30 μm. The size of the magnetic material is determined by SEM and refers to the longest dimension of the particle.

In some aspects, the carbon layer, such as a GUITAR layer has an average thickness of from 0.5 μm to 15 μm, such as from 1 μm to 10 μm, as determined by SEM.

The carbon layer may be any type of carbon suitable for use as a biosensor and/or functionalizing to conjugate a nucleic acid moiety, such as a DNA or RNA moiety, to the particle. In some aspects, the carbon layer comprises graphite and may comprise, consist essentially of, or consist of, graphite from the University of Idaho thermolyzed asphalt reaction (GUITAR).

The carbon layer is functionalized with a functional group suitable to conjugate a nucleic acid moiety to the particle. The functional group may be a carboxylate (—CO₂alkyl), carboxyl (CO₂H), amine (NH₂), hydroxyl (OH) for example via a silane coupling, or amino-thiol moiety. In some aspects, the functional group is a carboxylate or carboxyl moiety.

In certain aspects, the carbon layer is functionalized by treating the layer with 4-aminobenzoic acid such that the amine moiety attaches to the carbon layer (for example, by covalent and/or electrochemical bonds) and the carboxylic acid moiety is available to conjugate to the nucleic acid moiety.

The nucleic acid moiety may be any single stranded nucleic acid moiety, such as a single-stranded DNA (ss-DNA) or RNA molecule, suitable for use in the technology. The nucleic acid may of any length suitable for use in the disclosed technology. In some aspects, the nucleic acid moiety comprises from 10 bases to 1,000 bases or more, such as from 15 bases to 500 bases, or from 20 bases to 500 bases. In certain aspects, the nucleic acid moiety comprises from 20 bases to 50 bases. In other aspects, the nucleic acid moiety is from 200 bases to 500 bases. In certain examples, the nucleic acid moiety is a ssDNA comprising from 10 bases to 500 bases, such as from 20 bases to 50 bases, or from 200 bases to 500 bases.

In some aspects, the nucleic acid moiety is selected to be a single stranded nucleic acid moiety that is complementary to a target nucleic acid moiety. In certain aspects, the nucleic acid moiety is a ss-DNA that is complementary to a target or analyte DNA moiety. In some aspects, the nucleic acid moiety, such as a ss-DNA or RNA moiety, is a PCR primer and the disclosed magnetic particle is used in a PCR process. In other aspects, the ss-DNA or RNA is a DNA or RNA probe that is selected to be complementary to a nucleic acid target or analyte of interest.

### IV. SYNTHESIS

The GUITAR-coated iron particles are prepared by a Chemical Vapor Deposition (CVD) process. μ-Fe particles and diatomaceous earth are mixed and subjected to an initial CVD process using a suitable precursor at a temperature suitable for CVD under an inert atmosphere, such as nitrogen. Suitable precursors include, but are not limited to, condensed phase or volatile organic compounds, for example, vegetable oil; alkanes, such as hexane, heptane, etc.; acetonitrile; natural gas; petroleum products, such as petrol or diesel; roofing tar; clarified butter; or any combination thereof. The temperature may be from 750° C. to 1000° C., such as from 800° C. to 950° C. or from 850° C. to 950° C.

The resulting particles are screened to a suitable size, such as less than 50 μm, less than 40 μm, less than 30 μm, or less than 28 μm. The particles then are used in a second CVD process comprising a second precursor material, such as vegetable oil. After the second CVD process, particles that have exposed iron (for example, as determined by Prussian blue formation) are separated and discarded, along with non-magnetic particles of diatomaceous earth.

The GUITAR-coated iron particles are functionalized with a functional group suitable for nucleic acid conjugation, such as a carboxyl moiety. In some aspects, a solution of 4-aminobenzoic acid, an inorganic base, a nitrite salt, and an acid is prepared. The inorganic base may be any suitable base, such as a hydroxide base, for example sodium hydroxide or potassium hydroxide, or an ammonium base, such as ammonium carbamate. The nitrite salt may be any suitable nitrite salt, such as sodium nitrite or potassium nitrite. The acid may be any suitable acid, such as a mineral acid, for example, HCl.

A second mixture is prepared, comprising the magnetic particles and a detergent. The detergent may be any detergent suitable for facilitating functionalization of the GUITAR-coated magnetic particles. Exemplary detergents include, but are not limited to, sodium dodecylbenzene sulfonate (SDBS), triton x-100 (t-Octylphenoxypolyethoxyethanol or Polyethylene glycol tert-octylphenyl ether), sodium benzene sulfonate, or a combination thereof.

After functionalization, the nucleic acid moiety, such as a ss-DNA or ss-RNA moiety, is conjugated to the particles. Typically, the nucleic acid moiety is modified with a functional group suitable for conjugating to the functionalized GUITAR-coated magnetic particles. In some aspects, the nucleic acid moiety contains an amine group suitable for conjugating to a carboxyl group on the coated iron particle. In some aspects, the nucleic acid moiety and the functionalized magnetic particles are combined in the presence of a suitable coupling agent, such as a carbodiimide reagent, for example EDC or DCCI.

### V. APPLICATIONS

The disclosed magnetic particles can be used in any application that requires separation of a component of a mixture or a reagent, such as a catalyst. In some aspects, the disclosed magnetic particles are used in an application that require DNA or RNA detection, such as, but not limited to, viral pathogen detection, environmental surveillance, and/or food quality applications. The disclosed particles are particularly useful where a low limit of detection and/or rapid detection is desired, and could also be applied to isothermal amplification techniques. Also, the disclosed particles are useful when a PCR process is not available but rapid, low-level detection is desired.

Additionally, the disclosed magnetic particles are useful in DNA and/or RNA synthesis applications, such as when there is a desire to rapidly synthesize large quantities of ssDNA or ssRNA.

Other applications include, but are not limited to, biosensors comprising biomolecules such as enzymes, antibodies and/or proteins. In some aspects, the biomolecule is conjugated to the surface of the disclosed magnetic particles, such as by a suitable technique disclosed herein. Additional information concerning conjugating biomolecules to a carbon layer can be found in Smith, et al. A Comprehensive Review of the Covalent Immobilization of Biomolecules onto Electrospun Nanofibers, Nanomaterials 2020, vol. 10(11), 2142, which is incorporated herein by reference in its entirety.

Alternatively, the disclosed magnetic particles can be conjugated to a catalyst. In some aspects, the disclosed magnetic particles allow the catalyst to be easily removed from a reaction mixture. The catalyst can be any moiety suitable to catalyze a reaction. The catalyst may be a biomolecule, such as an enzyme, an inorganic catalyst, or an organic catalyst. The catalyst may be a homogeneous catalyst or a heterocatalyst.

In other aspects, the disclosed magnetic particles are useful for applications concerning determining or analyzing binding constants, for example, in molecule-nucleic acid interactions. In one aspect, the disclosed magnetic particles are useful for analyzing molecule-DNA interactions for cancer treatment.

And in further aspects, the disclosed magnetic particles are useful for electroanalytical applications, because the particles are electrochemically active.

### VI. EXAMPLES

### Example 1

### Coating the Iron Microparticles With GUITAR

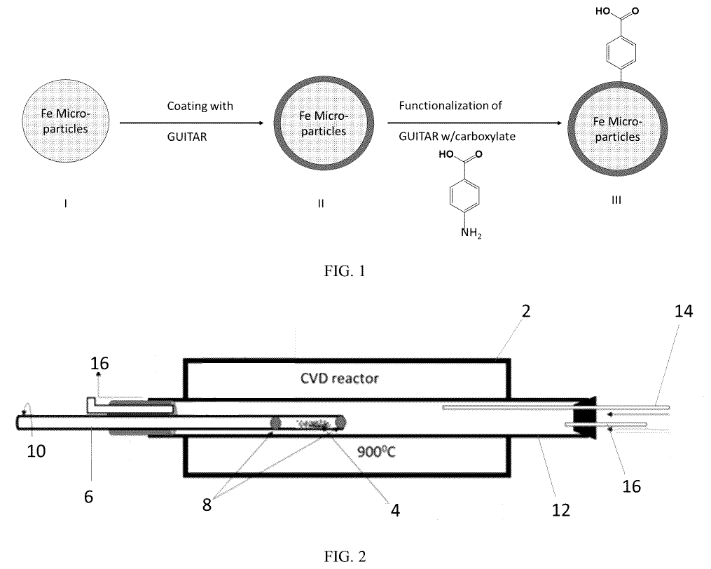

Chemical vapor deposition (CVD) was used to prepare the GUITAR-coated μ-Fe particles. The synthesis was based on a modification of the procedure disclosed by Kabir et al. (The Sp2-Sp3 Carbon Hybridization Content of Nanocrystalline Graphite from Pyrolyzed Vegetable Oil, Comparison of Electrochemistry and Physical Properties with Other Carbon Forms and Allotropes. Carbon 2019, 144, 831-840). Briefly, 2 g of μ-Fe particles (FIG. 1, Compound I) and 200 mg of diatomaceous earth were mixed in a crucible and subjected to an initial CVD process using vegetable oil as a precursor at 900° C. for 25 minutes under nitrogen atmosphere. After cooling, agglomerated particles were separated with a 28 μM mesh filter and then discarded.

FIG. 2 is a schematic diagram of a CVD reactor 2 suitable for use in a second CVD coating process. The <28 μm particles 4 were loaded into a small diameter (one inch) quartz tube 6 with and retained between quartz wool plugs 8. Arrow 10 indicates that the quartz tube 6 can rotate. The one inch quartz tube 6 was manually rotated in a larger 3″ diameter quartz tube 12 under vegetable oil injection 14 under flowing N₂ gas 16 for 20 minutes.

After cooling to room temperature, the particles were removed, and the coated iron particles (FIG. 1, Compound II) were collected using magnets. The GUITAR-coated diatomaceous earth particles were discarded. Any batch that exhibited exposed iron surface was rejected.

### Evaluation of Surface Coating of GUITAR

To evaluate defects of the GUITAR coatings that allow for electrolyte penetration to the Fe substrate, a sample of the coated particles was added to 25 mM K₃Fe(CN)₆ solution at pH 4. Exposed Fe is apparent by the formation of Prussian blue in the sequence shown below.

Fe⁰+2H⁺+2Cl⁻→Fe²⁺+2Cl⁻+H₂↑

Fe²⁺+[Fe(CN)₆]³⁻→Fe³⁺+[Fe(CN)₆]⁴⁻

4Fe³⁺+3[Fe(CN)₆]⁴⁻→Fe₄[Fe(CN)₆]₃ (Prussian blue)

Fully coated particles do not allow for the formation of Prussian Blue. Any batch that exhibited Prussian blue formation by visual inspection was rejected.

### Scanning Electron Microscopy

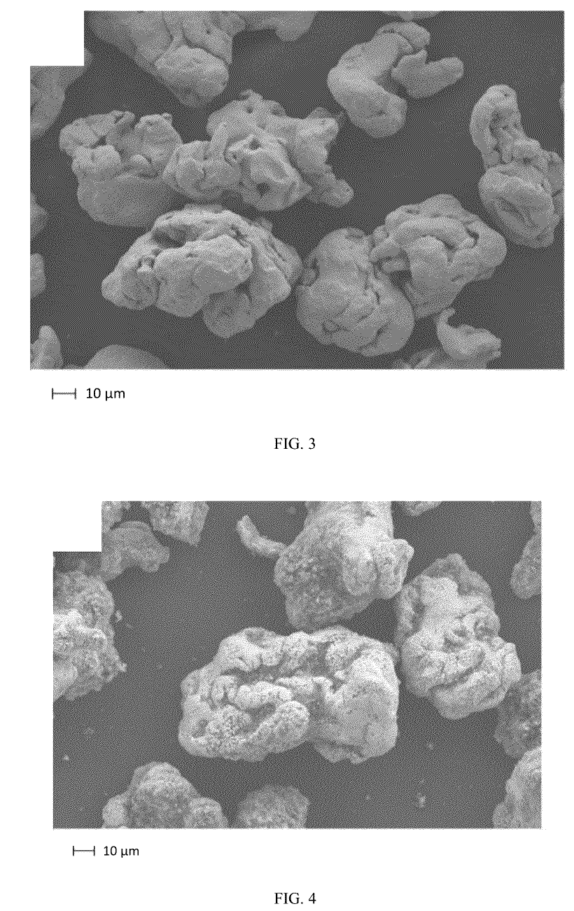

Scanning electron microscopy—energy dispersive spectroscopy (SEM-EDX) was used to investigate the elemental composition of the surfaces of μ-Fe (compound I) and GUITAR coated μ-Fe particles (compound II). Elemental composition at the surface of the μ-Fe, compound I showed the presence of iron and oxygen (FIG. 3). In contrast, the surface composition of compound II exhibited about 96% carbon with no oxygen detected (FIG. 4). Accordingly, the SEM images confirmed that GUITAR adhered to the μ-Fe substrate.

### Example 2

### Functionalizing the GUITAR Layer

Surface functionalization of GUITAR/μ-Fe particles with carboxylate groups was based on a modified literature procedure by Jahan et al. (Structure-Directing Role of Graphene in the Synthesis of Metal-Organic Framework Nanowire. J. Am. Chem. Soc. 2010, 132 (41), 14487-14495). A salt solution consisting of 960 mg 4-aminobenzoic acid, 280 mg sodium hydroxide, 526 mg NaNO₂ and 80 mL water was maintained at a temperature of 0-4° C. 5 mL of 6.4 M HCl solution was added to this solution and stirred for 45 minutes at 0-4° C. A separate mixture of 2 g of freshly GUITAR coated μ-Fe particles in 20 mL de-ionized water (DI H₂O) with 1% (w/w) sodium dodecylbenzene sulfonate (SDBS) was gently swirled while maintaining temperature at 0-4° C. The salt solution was added and stirred for 4 hours at 0-4° C. and allowed to come to room temperature for another 4 hours. This functionalized magnetic particle was washed 5× using a magnet and adjusted to pH 4 with concentrated HCl followed by further washing with ethanol and acetone. The functionalized particles (benzoic acid-GUITAR/μ-Fe, FIG. 1, Compound III) were dried at room temperature and stored at 4° C. for further use.

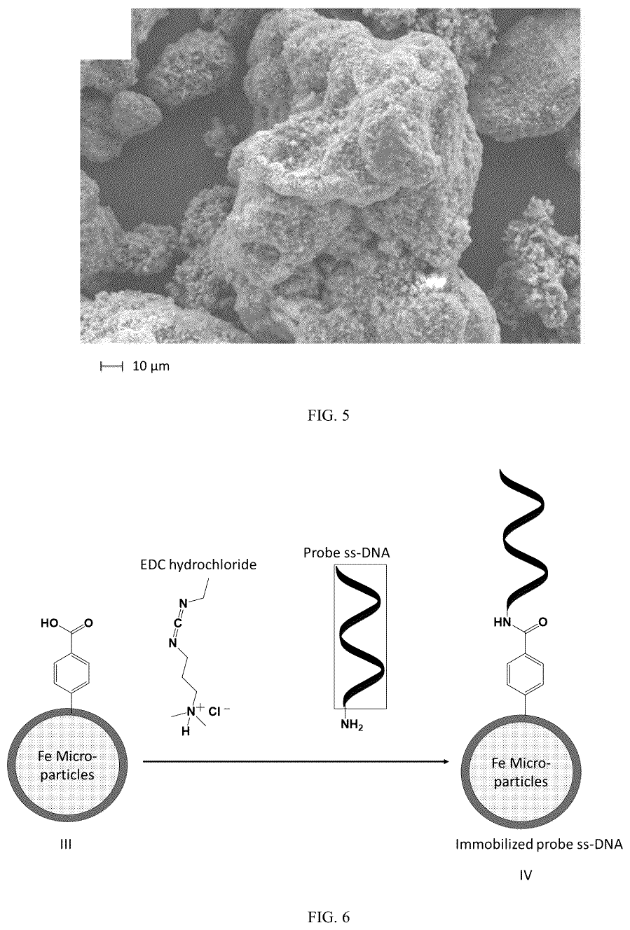

The SEM image of Compound III is shown in FIG. 5. Compound III contained about 3 atomic % of oxygen from the introduction of benzoic acid groups. Table 1 provides the elemental compositions of compounds I, II, and III from Examples 1 and 2 in tabular form. The energy dispersive x-ray analysis (EDX) results were for 6 runs each. The EDX confirmed the introduction of oxygen groups on to the surface of GUITAR of compound II.

TABLE 1
The elemental compositions of compounds I, II,
and III from Examples 1 and 2

| | Atomic % | | |
|---|---|---|---|
| Compound | Carbon | Iron | Oxygen |
| I | 0.41 | 98.42 | 1.17 |
| II | 96.41 | 3.59 | 0.00 |
| III | 94.24 | 3.02 | 2.74 |

### Example 3

### Conjugating the ss-DNA with Particles

Attachment of the probe single-strand (ss) DNA was through an amide linkage to Compound III. The 5′ amine modified single stranded oligonucleotide sequence used in this example is SEQ ID NO. 1.

(SEQ ID NO: 1)
/5AmMC6/5′-GTA GGC TGA GGT AGG GAG AAT GGG AGG TAC
ACC AGT TAG C-3′

The 5 nanomoles (63.7 μg) of the probe was added to a suspension of 100 mg of Benzoic acid-GUITAR/Fe microparticles (Compound III) in 1 mL pH 6.0, 0.1M MES buffer with 110 μL of 1M N-ethyl-N′-(3-dimethylaminopropyl) carbodiimide hydrochloride (EDC) coupling agent and gently vortexed for 6 hours. This gives Compound IV (FIG. 6). The suspension was collected with a magnet and washed three times with 0.1M HEPES buffer at pH 7.2. Approximately 45% (2.25 nanomoles) of the ssDNA was immobilized on 100 mg of Compound IV as determined by UV-vis absorbance measurements with the SpectraMax QuickDrop spectrophotometer.

### Example 4

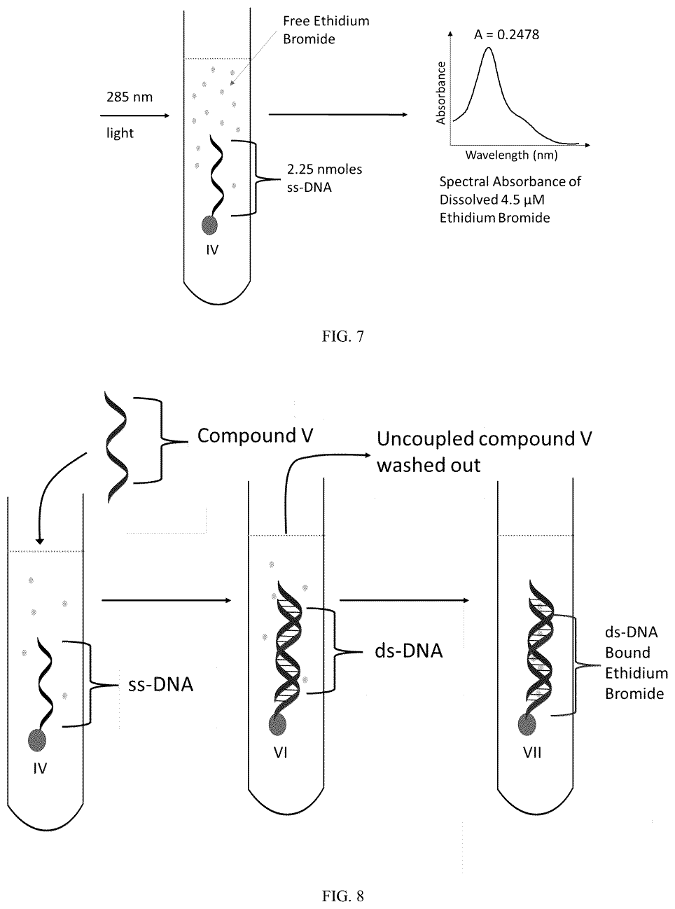

Compound IV particles (from 100 mgs Compound III modified with 2.25 nmoles ss-DNA) were dissolved into an ethidium bromide solution comprising 5.4 μM ethidium bromide, 0.1 M HEPES buffer pH 7.2, and 25 mM MgCl₂ to give an approximate total volume of 3 mL. A Beer's Law absorption signal was produced by the free ethidium bromide in the solution. The absorbance reading at 285 nm (for ethidium bromide, λ-max=285 nm) was 0.2478 (FIG. 7).

### Example 5

### Identification of a Complementary Target Analyte

The capture and analysis of a target oligonucleotide analyte by a complementary ssDNA decorated microparticles (FIG. 6, Compound IV) can be conducted without or with PCR amplification. The former requires 15 minutes and achieves an LOD of 24 nM and the latter under 2 hours with 25 PCR cycles and produces an LOD of 5 pM.

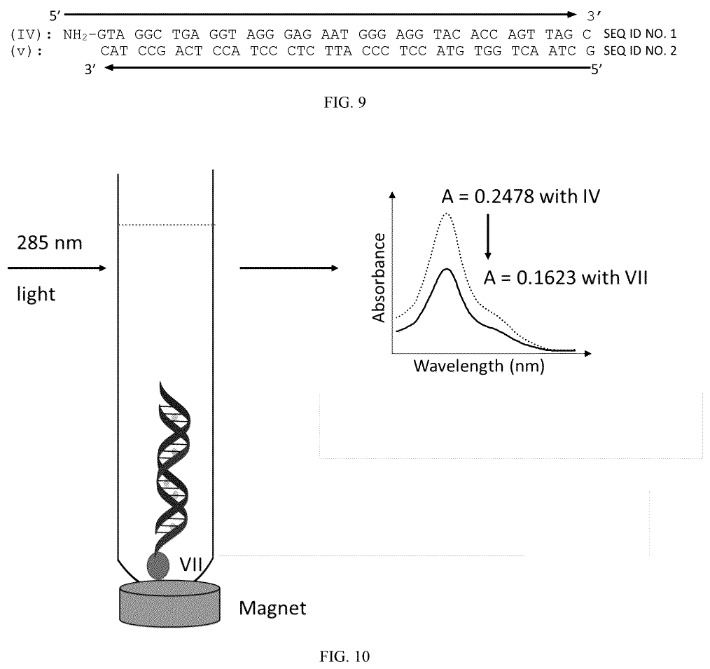

A 2.00 nmoles (24.1 μg, 2 μL of 1 mM) of a complementary target analyte of ss-DNA sequence (Compound V, SEQ ID NO: 2) was introduced into the reaction solution and allowed to hybridize with compound IV to give Compound VI (FIG. 8). The ds-DNA structure is shown in FIG. 9.

(SEQ ID NO: 2)
Compound V: 5′-GCT AAC TGG TGT ACC TCC CAT TCT CCC
TAC CTC AGC CTA C-3′

The Compound VI particles were collected with a magnet, washed with 0.1 M HEPES buffer at pH 7.2. About 10% (0.2 nmoles) of the ss-DNA target/analyte was unconjugated and was carried away in the wash. Without being bound to a particular theory, the unconjugated fraction was attributed to steric forces that did not allow for 1:1 mole ratio of Compound IV to the ss-DNA analyte Compound V. The washed Compound VI particles were transferred to a 4.5 μM ethidium bromide, pH 7.2, 0.1 HEPES buffer solution. The washing was required as DNA nucleotides (λ-max=260 nm) interfere with ethidium bromide spectral absorbance at 285 nm.

The aqueous ethidium bromide was allowed to intercalate into the double stranded DNA (FIG. 10, Compound VII). This action lowered the concentration of free aqueous ethidium bromide. The particles comprising ds-DNA with the intercalated ethidium bromide were separated from the rest of the solution with a magnet. And a Beer's Law analysis for free aqueous ethidium bromide in the remaining solution without the ds-DNA particles showed a decrease in the absorption signal. The absorbance reading at 285 nm (for ethidium bromide) was 0.1623 for Compound VII (FIG. 10). The drop in absorption from 0.25 for Compound VI to 0.16 for Compound VII is from the decrease in free ethidium bromide concentration due to the intercalation into ds-DNA.

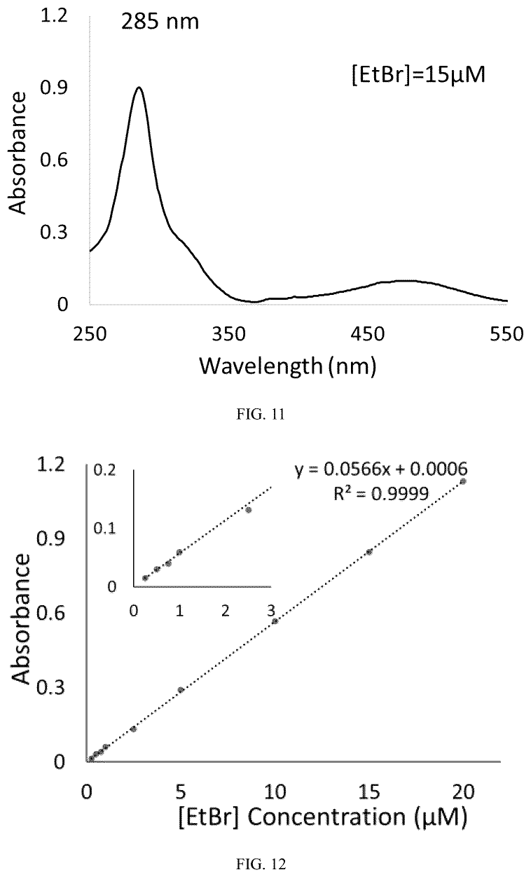

The concentration of free aqueous ethidium bromide was determined by a Beer's Law calibration curve at 285 nm. This is shown in FIGS. 11 and 12 with good linearity up to 20 μM ethidium bromide.

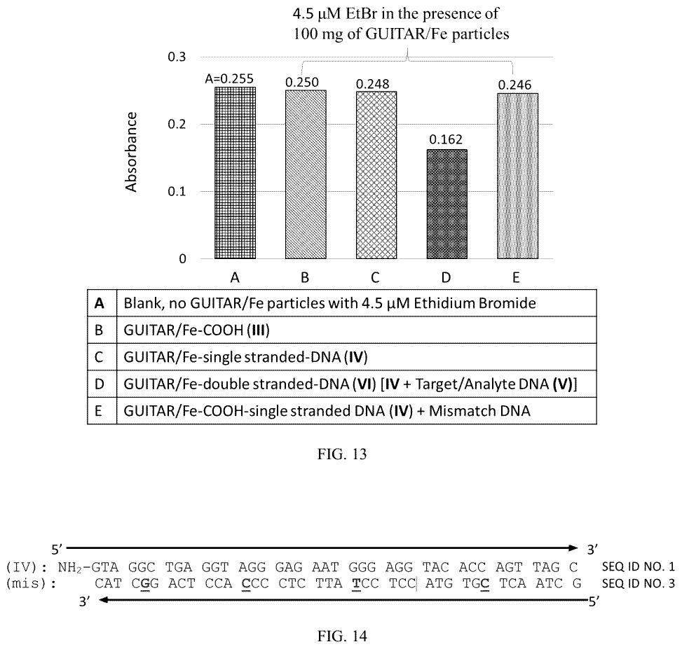

FIG. 13 provides ethidium bromide absorbance signals for different combinations of magnetic particles disclosed here and single- or double-stranded DNA. With respect to FIG. 13, the loss of Beer's Law absorbance signal for ethidium bromide in the presence of the double-strand DNA form of the GUITAR/Fe (Compound VI) is shown in Column D. The 3 controls in columns A, B and C maintained the absorbances of ca. 0.25 absorbance units. This signal drops to 0.16 when double stranded-DNA forms on the surface of the GUITAR/Fe particle (Compound VI) and is separated from the sample matrix with a magnet. The limit of detection of free aqueous ethidium bromide is 50 nM based on FIG. 12 using the 3σ/m method.

FIG. 13 shows the absorbances at 285 nm for ethidium bromide after the completion of the protocols shown in FIGS. 8 and 10 and any capture of ethidium bromide by immobilized DNA. Column D indicated that the formation of dsDNA on the compound IV particle reduced ethidium bromide absorbance relative to the controls in columns A-C. Column E indicates that the DNA capture protocol (FIGS. 8 and 10) with a mismatched sequence (SEQ ID NO. 3) did not capture aqueous ethidium bromide. This demonstrates the specificity of the technique for the complementary analyte ss-DNA. For this study, a ss-DNA was used that contained 4 mismatched pairs. The mismatches are shown below with the bases underlined.

(SEQ ID NO. 3)
5′ - GTC AAC TCG TGT ACC TCC TAT TCT CCC CAC CTC
AGG CTA C -3′

And FIG. 14 shows the mis-matched double-stranded DNA structure resulting from using SEQ ID NOS. 1 and 3.

For fully complementary DNA, Beer's law analysis at 285 nm on the supernatant after magnetic separation indicated a loss of absorbance of 0.0891±0.006 absorbance units (i.e., from 0.2498±0.004 in col. 3 to 0.1607±0.004 in col. 4) due to capture and removal of ethidium bromide from solution. These indicated that 8.4 nanomoles of ethidium bromide intercalated into 1.8 nanomoles of dsDNA in compound VI. This gave 8.6 base pairs per intercalated ethidium bromide. This is close to literature which report 4-5 base pairs per ethidium bromide.

### Second Experiment

In a second experiment, 2-5 nmoles of target (analyte) oligonucleotide sequence (Compound V) was allowed to hybridize to a 2.25 nmoles of complementary sequence bound to the surface of 100 mg of benzoic acid-GUITAR/μ-Fe (Compound IV). The oligonucleotide sequences used in this study were SEQ ID Nos. 1, 2 and 3 as previously described. Control experiments were performed using non-complementary DNA.

After 10 minutes of incubation at 60° C., the unhybridized (Compound IV) and/or hybridized (Compound VI) particles were collected with a magnet and washed with pH 7.2, 0.1 M HEPES buffer. The particles were transferred to an aqueous solution containing 22.5 nmoles of ethidium bromide buffered at pH 7.2. The ethidium bromide was allowed to intercalate into the dsDNA of Compound VI.

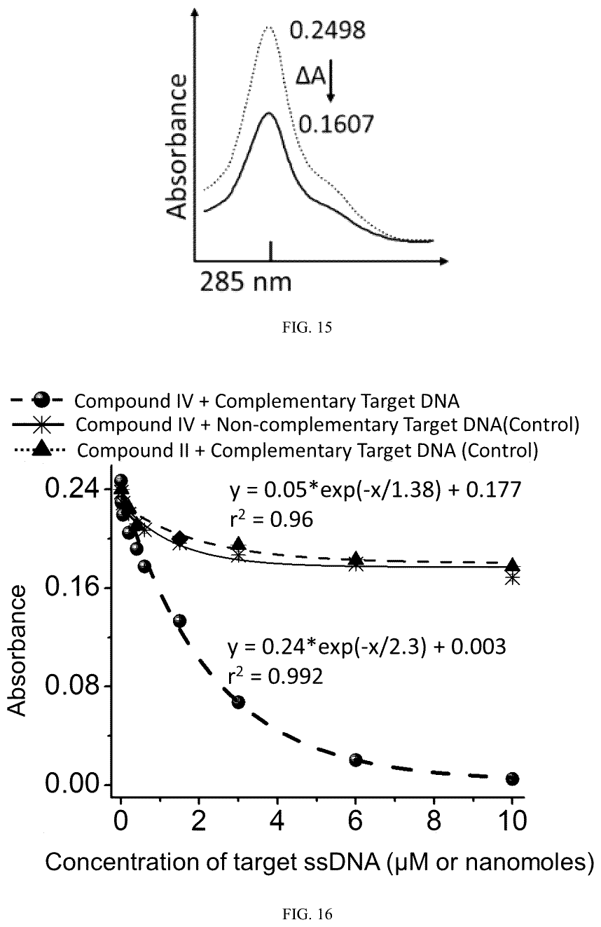

The particles in this solution were removed with a magnet. The supernatant was analyzed using a UV-Vis spectrophotometer and spectra are shown in FIG. 15. The dashed line (control) and solid line(experimental) showed a sharp decrease in the absorbance of ethidium bromide at its spectral maximum at 285 nm (ΔA). This was proportional to the quantity of captured target DNA. Estimates for the limit of detection (LOD) are presented below.

### Example 6

### Limit of Detection for Target ssDNA Without PCR

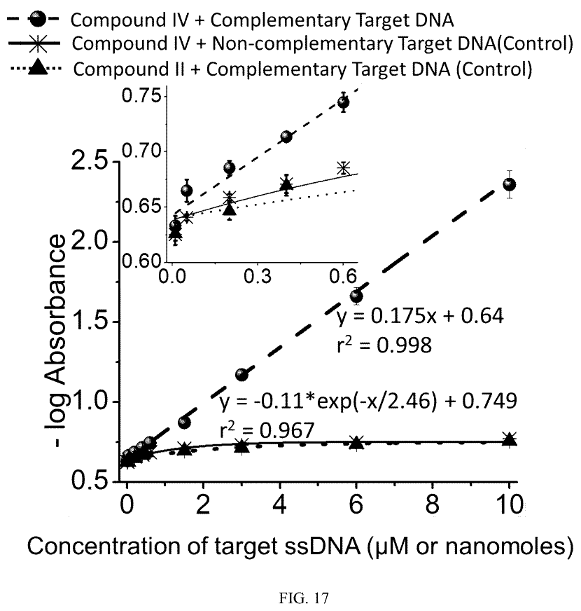

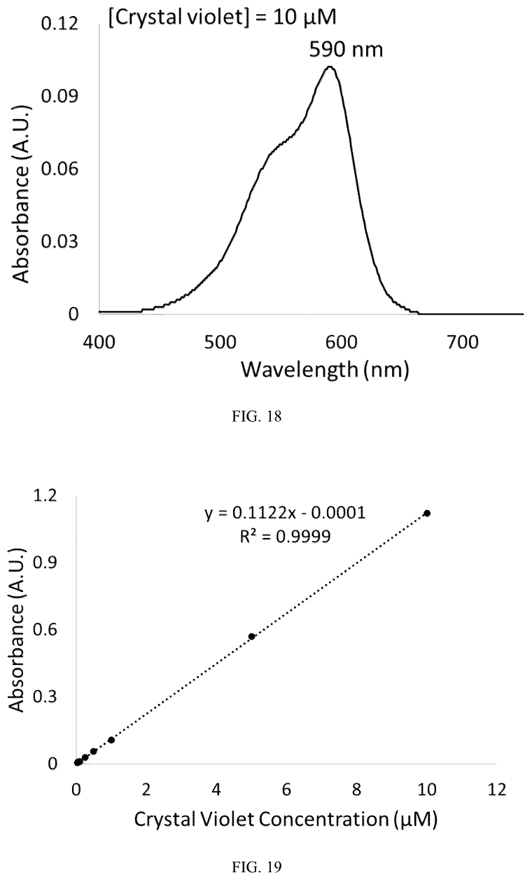

Concentration of analyte ssDNA was measured from the decrease in aqueous ethidium bromide absorbance after magnetic accumulation of VI. FIGS. 16 and 17 summarize the limit of detection (LOD) of the disclosed method when measuring the target 40-base SEQ ID NO. 2. The absorbance at 285 nm of 4.5 μM ethidium bromide was measured with the following conditions. Compound IV particles (500 mg) containing 11.25 nanomoles of probe ssDNA (also 11.25 μM) in 0.1 M HEPES pH 7.2 was added to 0.010 to 10.00 μM (or nanomoles) of target sequence (dashed line). Two controls consisting of mismatch target DNA (SEQ ID NO. 3) with Compound IV and a carbon-only surface with no probe ssDNA (compound II) are included in this study. The graphs in FIGS. 16 and 17 indicate that the controls behaved largely in the same way. There is a drop in absorbance from 0.25 to 0.16 that levels off above 1 μM. This may indicate that the loss of free aqueous ethidium bromide is from adsorption onto the surfaces of these particles with saturation above 1 μM. It is apparent there is a much greater drop when dsDNA forms giving compound IV as compared with the controls. The mismatch sequence and bare surface had similar absorbance responses. The best fit exponential functions are shown. The controls gave nonlinear responses below 0.2 nanomoles of complementary ssDNA or mismatch. This is shown in the inset FIG. 17. with error bars based on the standard deviation of three runs.

The exponential fit for free ethidium bromide absorbance in FIGS. 16 and 17 agrees with other aqueous dsDNA-intercalator systems. Also in FIG. 17, the relationship between analyte ssDNA concentration and log (A) was linear. The low concentration characteristics are shown in the inset. The estimate for LOD was 24 nM (0.87 μg) based on 3σ/m, where σ is estimated to be 0.0014 log (A) units at low concentrations of 0.01 and 0.02 μM. And the slope for the linear fit was 0.175 log(A)/μM. This was confirmed with t-test between the 25 nM data point from FIGS. 16 and 17 and the bare surface blank which indicated that these quantities were significantly different with a confidence level of 95%. This LOD could be used to detect viral pathogens with ssDNA genomes such as nanoviridae which threaten legume and banana crops. Other methods require 2+ hours for sample preparation and analysis. The disclosed method takes only 15 minutes.

### Example 7

### Using Alternative DNA Intercalators

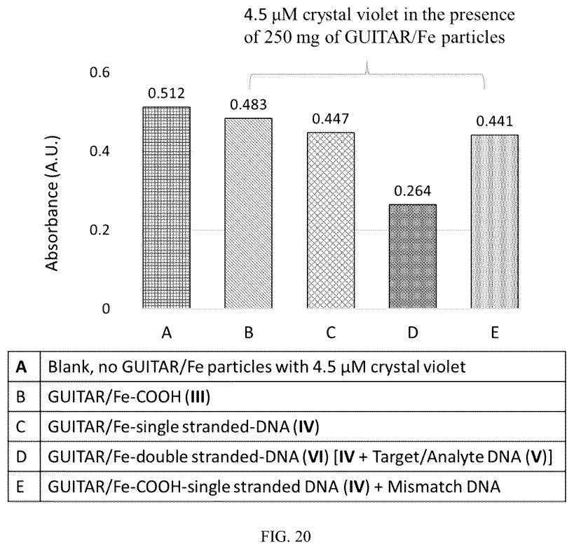

Other DNA intercalators are possible with this technique. With crystal violet (λ-max=590 nm) in place of ethidium bromide a limit of detection of 6 ng was attained, (3 ng for ethidium bromide). Crystal violet has an advantage over ethidium bromide in that its spectral absorbance does not overlap with DNA nucleotides, thus allowing for the elimination of the washing step (between Compound IV and Compound VI). Also, it allows for the use of less costly visible wavelength spectrometers. The result is shown in FIGS. 18 and 19. The limit of detection for crystal violet is lower than ethidium bromide which may be attributed to larger binding site sizes. Crystal violet binds to 10-14 bp vs. 4-5 bp for ethidium bromide. FIG. 20 summarizes the studies with the visible dye. In this application the calculated binding of compound VII is 30 bp per immobilized ssDNA. Crystal violet concentration was 4.5 μM in all columns.

### Example 8

### Evaluation of the System With the Polymerase Chain Reaction (PCR)

One important advantage of the GUITAR/Fe-based system is that the covalent bond between the ss-DNA and the particle surface is stable during the conditions required for the PCR amplification of a ss-DNA analyte strand.

Similar schemes based on gold nanoparticles (AuNPs), affinity binding (e.g. streptavidin-biotin interactions) and silica coated magnetic nanoparticles are not compatible with PCR. Attachment of DNA to AuNP is through sulfur bonds. However, these bonds are not stable under conditions required for PCR. Furthermore, Au-DNA conjugates decompose within hours due to both desorption of thiol-terminated DNA from the gold nanoparticle surface and chemical degradation of DNA in the presence of colloidal gold. Streptavidin-biotin is one of the strongest non-covalent interactions known, approaching the covalent bond strength. However, this strategy is limited by the size and stability of streptavidin, a large 66 kDa protein which denatures under conditions required for PCR (85° C.). Also, these beads are expensive. Silica is another sorbent material but cannot be used in the PCR scheme disclosed herein. Silica and/or silica coated magnetic materials absorbs dyes giving false-positives.

A significant feature of the GUITAR/Fe system is that compound IV is compatible with PCR (FIG. 8). This reduces the limit of detection to 2 pg with 25 PCR cycles from 3 ng without PCR. The lower limit of detection gives the ability to detect pathogens in clinical situations and rapid forensics analyses. Neither AuNP nor silica coated magnetic nanoparticles are capable of such low limits of detection.

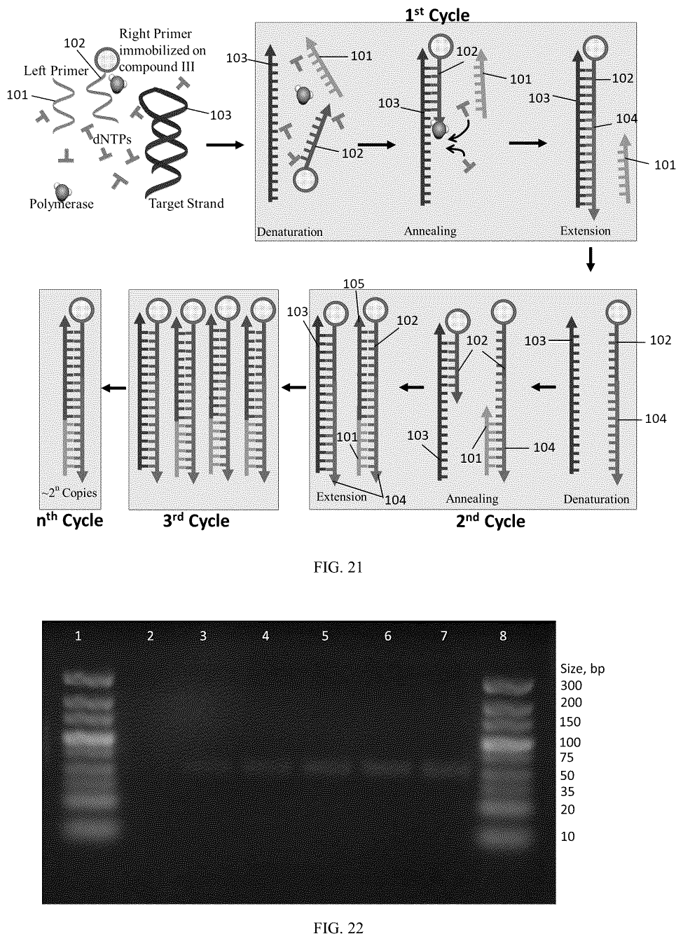

A variety of PCR strategies can be used with the disclosed particles.
[1] The particles can be used to isolate analyte ss-DNA from the bulk matrix. It can be denatured, and the target nucleic acid/analyte (compound V in FIG. 8) can be separated from immobilized nucleic acid (compound IV). PCR amplification and fluorescence detection of analyte can be performed by adding forward and reverse primer and Master-mix containing fluorescence dye. PCR amplification takes place only in the presence of target (analyte) nucleic acid.
[2] In another scheme shown in FIG. 21, the forward primer 102 (right primer) is immobilized to compound III and 25 mg is added to PCR vial containing reverse primer 101 (left primer), deoxyribose nucleotide triphosphate (dNTPS), and polymerase. In the presence of an analyte 103, PCR amplification takes place along with elongation of forward primer to form hybridized sequence (compound VI). This hybridized sequence can be detected via intercalation followed by absorption study.

This study demonstrates the robustness of the ss-DNA to GUITAR bond as other similar systems cannot withstand the conditions required for PCR. This has many possible applications including DNA capture, sequence-specific nucleic acid purification and enrichment, PCR clean-up, single strand generation.

To demonstrate the use of the disclosed particles for PCR, an oligonucleotide sequence was selected from the genome of Salmonella typhimurium (National Library of Medicine, GenBank: CP014358.1). Primer 3 software was used to design the target and primers for PCR reaction. The oligonucleotide sequences used for PCR are shown in Table 2 below. The amine-modified primer is immobilized to compound (III) (carboxylic functionalized GUITAR coated Fe micro-particles).

TABLE 2
Sequences used in the PCR process shown in FIG. 21

| | Nucleic acid sequences | |
|---|---|---|
| Label | (5′→3′) | SEQ ID NO. |
| Red (103) - Analyte/Target | GCT GTG GAA TCC GTT | SEQ ID NO. 4 |
| nucleic acid. (5 pM or more). | AGT GAA GTA ACC GAT | |
| | AAC GCC ACG GGA ATC | |
| | TCT | |
| Blue (102) - Amine modified | /5AmMC6/AGA GAT TCC | SEQ ID NO. 5 |
| right primer, attached to | CGT GGC GTT AT | |
| GUITAR/Fe as IV. | | |
| Underlined sequence is | | |
| complementary to the | | |
| Analyte underlined sequence | | |
| above. | | |
| Orange (101) - left primer | GCT GTG GAA TCC GTT | SEQ ID NO. 6 |
| (Primes complimentary strand | AGT GAA | |
| of target) | | |
| Green (104) - Extension of | ATC GGT TAC TTC ACT | SEQ ID NO. 7 |
| right primer (in blue) with | AAC GGA TTC CAC AGC | |
| addition of dNTPS to from | | |
| the complementary of | | |
| Analyte Sequence. | | |
| Purple (105) - extension of | GTA ACC GAT AAC GCC | SEQ ID NO. 8 |
| left primer (in orange) with | ACG GGA ATC TCT | |
| addition of dNTPs to form the | | |
| copies of target strand | | |

With respect to FIG. 21, in presence of a target nucleic acid, the immobilized primer extends to give a complementary of a target strand. The left primer then acts upon it so that the target is amplified in successive PCR cycle. In absence of target nucleic acid, no amplification takes place.

### PCR Methodology.

Temperature Program. The PCR temperature cycling program consisted of initial denaturation at 95° C. for 3 minutes, followed by varying (5-35) cycles of 95° C. for 30 seconds (denaturation), 58° C. for 30 seconds (annealing), and 72° C. for 30 seconds (extension). After the final PCR cycle, the holding time was 5 minutes at 72° C.

### Optimization of Number of PCR Cycles.

The PCR products were then assayed by gel electrophoresis on 2.5% agarose gel in 1×TAE buffer (0.04 mol/L tris-acetate+0.002 mol/L EDTS) was performed on PCR products of various (5-35) PCR cycles. This was done as follows; 2.5% agarose gel was cast in a gel tray equipped with 8-well comb. The comb was removed gently when the gel was cooled. 2 μL of PCR products from varying (3-35) PCR cycles was mixed with 2 μL of 6× blue gel loading dye. The final volume was brought to 12 μL by adding nuclease free water. Similarly, 20 μL, ultra-low range DNA ladder (ThermoFisher Scientific) was mixed with 2 μL of 6× blue gel loading dye. The final volume was brought to 12 μL by adding nuclease free water.

The gel was set in BioRad horizontal electrophoresis system. 5 uL of the sample was loaded in each well. Wells 1 and 8 contained DNA ladder solutions. Wells 2-7 contained PCR products solution. 75 V potential was applied for 45 minutes to perform gel electrophoresis. It was followed by ethidium bromide (0.5 μg/ml) staining for 30 minutes. It was then washed twice with DI water and photographed under UV light. The electrophoresis gel is shown in FIG. 22.

The optimum number of PCR cycles was determined as 25 (FIG. 22). Various number of PCR cycles were performed, and agarose gel electrophoresis was done. The gel was stained with ethidium bromide and the image was taken by illuminating the gel with UV light. With respect to FIG. 22:
Lanes 1 and 8 illustrate an ultra low range DNA ladder;
Lane 2 shows the result at 10 cycles;
Lane 3 shows the result at 15 cycles;
Lane 4 shows the result at 20 cycles;
Lane 5 shows the result at 25 cycles;
Lane 6 shows the result at 30 cycles; and
Lane 7 shows the result at 35 cycles.
   These indicated that 25 PCR cycles were the optimum for detection.

### Example 9

### Increasing the Speed of PCR Enrichment and Isolation

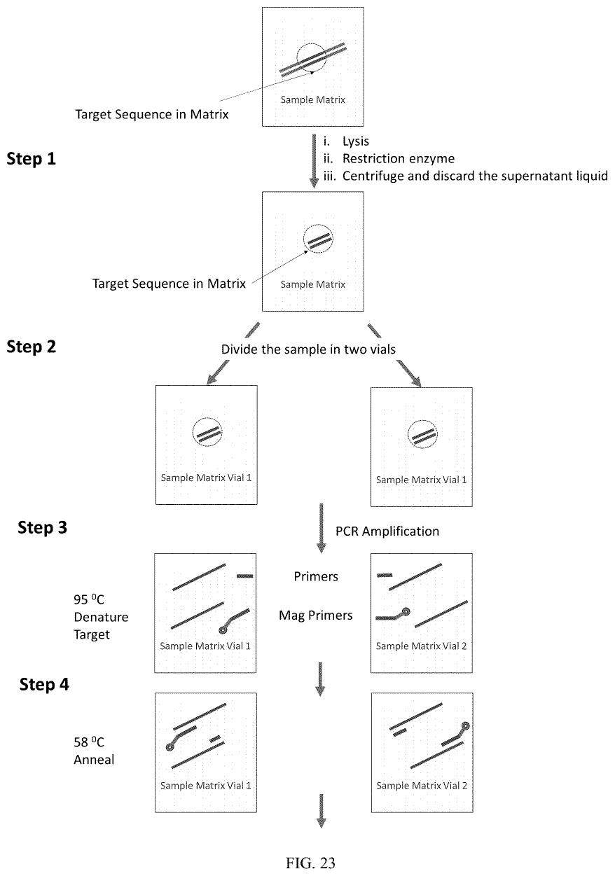

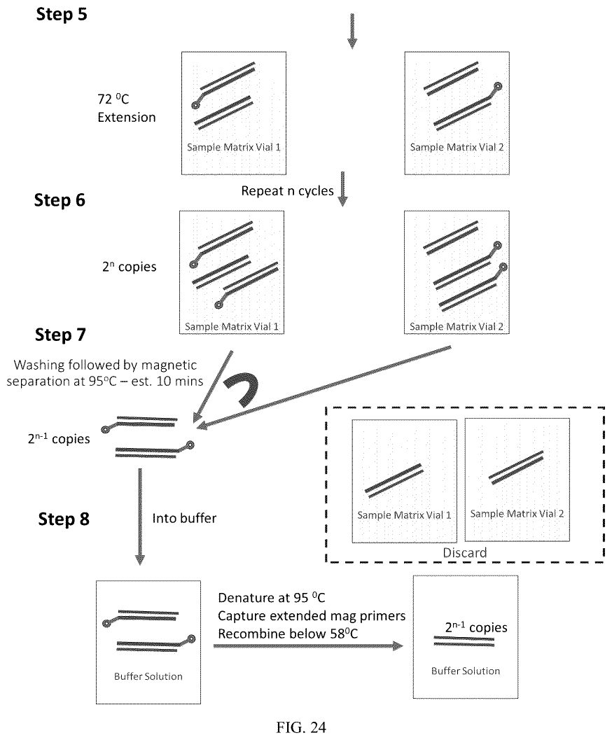

Present PCR methodologies require a total of 8-10 hours for the isolation and amplification of a target DNA sequence, typically 200 to 500 bp. The use of PCR amplification on primers immobilized on the disclosed magnetic particles decreases this time to approximately 2 hours, as illustrated in the procedure below and in FIGS. 23 and 24.

### Steps 1-2, Total 20 Minutes

Step 1—The cells containing the DNA sequence undergoes lysis, with the addition of restriction enzymes that isolate the target sequence.
Step 2—The sample is divided into 2 vials.
   Steps 3-6 total time 1.5 hours
Step 3—PCR amplification step. Add dNTPs to excess, isothermal amplification buffer(s), DNA polymerase and primers and immobilized primers magnetically susceptible particles. Then denature the sample at 95° C.
Step 4—Anneal at 58° C.
Step 5—Extension at 72° C.
Step 6—Repeat n cycles from Step 3 to 5.
   Steps 7-8, total time 10 minutes.
Step 7—At room temperature, apply magnet to vial the wash the magnetically immobilized dsDNA with distilled water. Discard the materials supernatant suspensions.
Step 8—Combine the magnetically susceptible particles from the 2 vials. Denature at 95° C., retain magnetically susceptible particles then pour off free-ssDNA suspensions into a combined vial. Allow this vial to cool to room temperature forming 2ⁿ⁻¹ copies of ds target DNA.

### Total Time 2 Hours.

### Example 10

### Nucleic Acid Sequence Detection: Detecting a Specific 48-Base Oligonucleotide Using the Polymerase Chain Reaction (PCR) on GUITAR/μ-Fe

PCR amplification of the analyte DNA can substantially lower the LOD for the technique above. However, implementing PCR into a rapid method for a target ssDNA analysis suffers from barriers. Perhaps the most significant is the separation of the amplified target ssDNA from the PCR reaction matrix. Present methods require more than 5 hours for target ssDNA isolation and purification with a total time of 8 hours and extensive instrumentation. Real time PCR (rtPCR or qPCR) requires a thermocycler with expensive and complex fluorescence optics. The GUITAR/μ-Fe system offers a pathway around those barriers. This magnetically susceptible system allows for rapid separation from the PCR matrix. Another advantage of this system is that it meets the need for a thermally stable linkage for the immobilization of ssDNA. The PCR conditions require raising the matrix above 90° C. in order to denature the dsDNA. Most microparticles, including magnetic polymer microparticles and Au nanoparticles (NP) are not PCR compatible. Polystyrene-based particles aggregate during the high temperature denaturation step and the thiol linkages on AuNP are not thermally stable. In general, PCR with immobilized ssDNA has not been fully realized until the technique disclosed herein.

The disclosed study demonstrated the coupling of PCR with the compound IV magnetic particles to detect a DNA sequence from the genome of Salmonella typhimurium. In food safety tests, the concentration of pathogen can be down to pM. Furthermore, the matrix may contain large concentrations of non-pathogenic DNA. This requires a specific isolation and amplification of the target sequence. To demonstrate this, a 48 bp sequence of Salmonella typhimurium was selected for isolation (Table 3). A complete genome of Salmonella typhimurium was obtained from National Library of Medicine, GenBank: CP014358.1. Primer 3 software was used to design the strand target of 48 bp for our PCR reaction and 20 and 21 bp primers (Table 3).

TABLE 3
Oligonucleotide sequences used for PCR studies

| Label | Nucleic acid sequences (5′ to 3′) | SEQ ID NO. |
|---|---|---|
| 48 base Analyte/Target | GCT GTG GAA TCC GTT AGT GAA | SEQ ID NO. 9 |
| nucleic acid. (5 pM or | GTA ACC GAT AAC GCC ACG GGA | |
| more) | ATC TCT | |
| 20 base Amine | /5AmMC6/AGA GAT TCC CGT GGC | SEQ ID NO. 10 |
| modified | GTT AT | |
| right primer, attached | | |
| to | | |
| GUITAR/Fe as IV | | |
| 21 base Left primer | GCT GTG GAA TCC GTT AGT GAA | SEQ ID NO. 11 |
| (Primer complimentary | | |
| strand of target) | | |
| 52 base Random | GCT AAC TGG TGT ACC TCC CAT | SEQ ID NO. 12 |
| Sequence 1 | TCT CCC TAC CTC AGC CTA CTC | |
| | TCC TCC TCA A | |
| 45 base Random | TGG AGA CGT AGG GTA TTG AAT | SEQ ID NO. 13 |
| Sequence 2 | GAG AGT GGA GAT GGG AGT AGT | |
| | TGG | |
| 26 base Random | TAT CAT ATG AAT CGC TGC TGG | SEQ ID NO. 14 |
| Sequence 3 | GCG CT | |

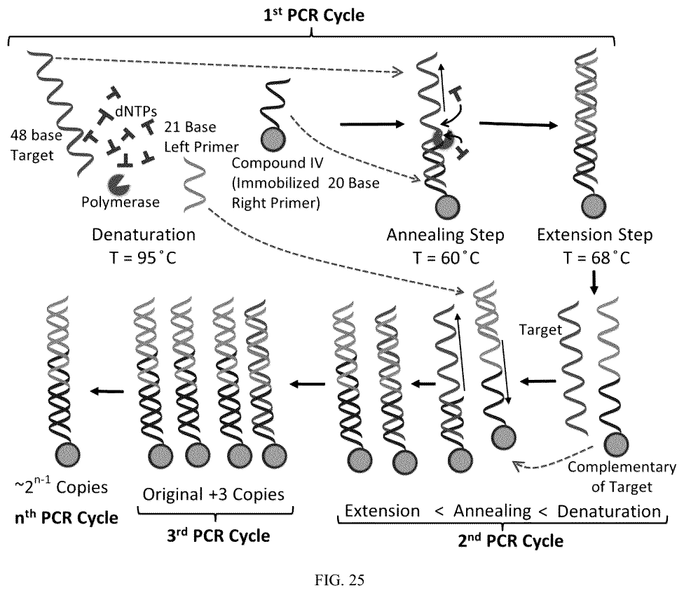

To conduct PCR with the functionalized magnetically susceptible particles, the amine modified 20 bp right primer sequence was immobilized onto benzoic acid functionalized particles (Compound III). This provided the ability to specifically prime and amplify the 48 bp target from a solution matrix as an immobilized product. Four studies were conducted, (1) the 48 bp target sequence, (2) 48 bp target sequence with mixture of random sequences varying from 26 to 52 bp (Table 3) with target, (3) the random sequences without target and (4) target without the immobilized 20 base right primer but with 21 base left primer (Table 3). FIG. 25 illustrates the PCR scheme with the magnetically susceptible particles.

The PCR steps and details are as follows. In the first cycle, the target strand (red, 1.85 pg or 5 pM up to 5 nM, 25 μL reaction volume) annealed to the right primer (blue, 2.1 μg or 12.5 μM) on the particle (compound IV, 12.5 mg). The deoxynucleoside triphosphate (dNTPs) concentration was 240 μM, and DNA polymerase was 1 unit. The immobilized right primer (blue) recognized the 48 base ssDNA target and polymerase extended the 28 base complementary sequence (green). In the second PCR cycle, the newly generated DNA was denatured, and the process repeated along with 21 base left primer (12.5 μM) PCR mode, generating two new copies; one was immobilized (green) while the other was not linked to the particle (black). The process repeats with subsequent cycles until compound IV was completely consumed.

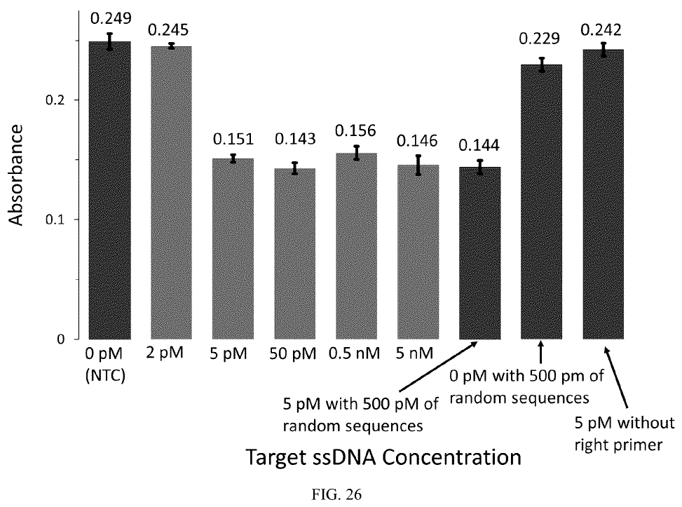

The optimum PCR cycle was determined to be 25 cycles after which, the particles were magnetically collected and washed 5 times. The ethidium bromide absorbance protocol was applied as described herein. Target ssDNA concentrations were detected from 0 to 5000 pM. Two of the control studies included the presence of 3 random sequences ranging from 52 to 26 bases (Table 3) each at 500 pM, 100× over the target LOD. FIG. 26 highlights the loss of absorption signal for 3 mL of 4.5 μM ethidium bromide after the protocols shown in FIG. 13. As the loss of this signal was minimal at 2 pM relative to the blank (0.249 absorbance units), the limit of detection was estimated to be 5 pM or 1.85 picograms of target ssDNA for that 25 μL reactor volume. This gave 0.152 absorbance units per 100 mg of compound IV. The absorbance signal losses from 5 to 5000 pM remained constant as the concentration of immobilized right primer is the limiting reagent. Also in FIG. 26, the controls with random ssDNA sequences did not affect the signal with 0 and 5 pM target ssDNA. The absence of immobilized primer also did not affect the ethidium bromide absorbance signal.

The system demonstrated in this study reduced the total time of analysis relative to qPCR methods with LOD's that match present techniques. The overall processing time was 2 vs. 8-10 hours for qPCR. Furthermore, it is expected to reduce costs by forgoing separation steps, simplified instrumentation and through the use of label-free probe sequences. When compared to present qPCR methods, using the same primer and template the LOD was 1-2 pM vs. 5 pM (1.85 pg) with technique of this contribution.

Luminex beads have also been used to measure specific DNA strands albeit with much longer processing time. Such beads capture target DNA by hybridization. That target DNA confers a fluorescent label on the bead surface with an LOD of 10-50 pg. In the present form, the coupling of PCR to this technique remained semi-quantitative, i.e. positive or negative but with low limits of detection and relative rapidity. It is important to note that this binary answer is shared with all PCR systems. The system disclosed herein improves on present qPCR methods for many important applications, especially considering the simplified workflow, analysis time, probe costs, and a simplified detector. These may include viral pathogen detection, environmental surveillance or food quality applications.

In view of the many possible embodiments to which the principles of the disclosed technology may be applied, it should be recognized that the illustrated embodiments are only preferred examples of the disclosure and should not be taken as limiting the scope of the disclosure. Rather, the scope of the disclosure is defined by the following claims. We therefore claim as our disclosure all that comes within the scope and spirit of these claims.

We claim:

1. A composition, comprising:
a magnetic material particle comprising an outer surface; and
a carbon layer located on the outer surface of the magnetic material particle.

2. The composition of claim 1, wherein the magnetic material particle is an iron particle.

3. The composition of claim 1, wherein the magnetic material particle has an average particle size of from 5 μm to 100 μm.

4. The composition of claim 3, wherein the average particle size of the magnetic material particle is from 15 μm to 40 μm.

5. The composition of claim 1, wherein the carbon layer covers greater than 95% of the surface of the magnetic material particle.

6. The composition of claim 1, wherein the carbon layer comprises graphite.

7. The composition of claim 6, wherein the graphite is graphite from the University of Idaho thermolyzed asphalt reaction (GUITAR).

8. The composition of claim 1, wherein the magnetic material particle, together with the carbon layer, has an average particle size of from 15 μm to 40 μm.

9. The composition of claim 1, wherein the carbon layer is functionalized with 4-amino benzoic acid.

10. The composition of claim 1, further comprising a single stranded nucleic acid.

11. The composition of claim 10, wherein the single stranded nucleic acid has a length of from 10 bases to 1,000 bases.

12. The composition of claim 11, wherein the length of the single stranded nucleic acid is from 20 bases to 50 bases, or from 200 bases to 500 bases.

13. The composition of claim 10, wherein the single stranded nucleic acid is single stranded DNA.

14. The composition of claim 10, wherein the single stranded DNA is a PCR primer.

15. The composition of claim 10, wherein the single stranded DNA is complementary to a target or analyte DNA moiety.

16. The composition of claim 1, wherein the composition is a particulate composition where each particle of the particulate composition comprises:
an iron particle substantially coated with a carbon layer, the carbon layer comprising graphite from the University of Idaho thermolyzed asphalt reaction (GUITAR); and
single stranded DNA conjugated to the carbon layer through an amino benzoic acid moiety on the carbon layer, the single stranded DNA having a length of from 20 bases to 500 bases;
wherein each particle of the particulate composition has an average particle size of from 1 μm to 30 μm.

17. A method, comprising:
forming a mixture comprising a magnetic particle and a single stranded nucleic acid molecule in a solution;
adding an intercalating agent to the mixture;
separating the magnetic particle from the solution; and
measuring the UV absorbance of the intercalating agent in the solution;
wherein the magnetic particle comprises
a magnetic material particle comprising an outer surface;
a carbon layer located on the outer surface of the magnetic material particle; and
a single stranded nucleic acid moiety conjugated to the carbon layer, the single stranded nucleic acid moiety being complementary to the single stranded nucleic acid molecule.

18. A method, comprising:
forming a mixture comprising a magnetic particle and a first PCR primer moiety, and a target nucleic acid strand; and
performing a PCR process using the mixture;
wherein the magnetic particle comprises a magnetic material particle comprising an outer surface, a carbon layer located on the outer surface of the magnetic material particle, and a second PCR primer moiety.

19. A method for making a plurality of particles according to claim 1, the method comprising:
exposing magnetic material particles and silica particles to a first CVD process in the presence of a first CVD precursor;
separating particles of a desired size from agglomerated particles;
exposing the particles of a desired size to a second CVD process in the presence of a second CVD precursor to form a carbon layer on the magnetic material particles; and
separating magnetic particles that are coated with the carbon layer from the silica particles to form the plurality of particles according to claim 1.

20. The method of claim 19, further comprising:
functionalizing the carbon layer on the plurality of particles by treating the particles with 4-amino benzoic acid in the presence of an inorganic base; and
conjugating the plurality of particles to a single stranded nucleic acid moiety.


SEQUENCE LISTING (14 sequences)

SEQ ID NO: 1 - DNA, 40 bp, synthetic construct
  source 1..40: mol_type = other DNA
  modified_base 1: mod_base = OTHER; note = guanine with 5-prime 6-aminohexyl hydrogen phosphate
gtaggctgaggtagggagaatgggaggtacaccagttagc

SEQ ID NO: 2 - DNA, 40 bp, synthetic construct
  source 1..40: mol_type = other DNA
gctaactggtgtacctcccattctccctacctcagcctac

SEQ ID NO: 3 - DNA, 40 bp, synthetic construct
  source 1..40: mol_type = other DNA
gtcaactcgtgtacctcctattctccccacctcaggctac

SEQ ID NO: 4 - DNA, 48 bp, synthetic construct
  source 1..48: mol_type = other DNA
gctgtggaatccgttagtgaagtaaccgataacgccacgggaatctct

SEQ ID NO: 5 - DNA, 20 bp, synthetic construct
  source 1..20: mol_type = other DNA
  modified_base 1: mod_base = OTHER; note = adenine with 5-prime 6-aminohexyl hydrogen phosphate
agagattcccgtggcgttat

SEQ ID NO: 6 - DNA, 21 bp, synthetic construct
  source 1..21: mol_type = other DNA
gctgtggaatccgttagtgaa

SEQ ID NO: 7 - DNA, 30 bp, synthetic construct
  source 1..30: mol_type = other DNA
atcggttacttcactaacggattccacagc

SEQ ID NO: 8 - DNA, 27 bp, synthetic construct
  source 1..27: mol_type = other DNA
gtaaccgataacgccacgggaatctct

SEQ ID NO: 9 - DNA, 48 bp, synthetic construct; identical to SEQ ID NO: 4
  source 1..48: mol_type = other DNA

SEQ ID NO: 10 - DNA, 20 bp, synthetic construct
  source 1..20: mol_type = other DNA
  modified_base 1: mod_base = OTHER; note = adenine with 5-prime 6-aminohexyl hydrogen phosphate
agagattcccgtggcgttat

SEQ ID NO: 11 - DNA, 21 bp, synthetic construct
  source 1..21: mol_type = other DNA
gctgtggaatccgttagtgaa

SEQ ID NO: 12 - DNA, 52 bp, synthetic construct
  source 1..52: mol_type = other DNA
gctaactggtgtacctcccattctccctacctcagcctactctcctcctcaa

SEQ ID NO: 13 - DNA, 45 bp, synthetic construct
  source 1..45: mol_type = other DNA
tggagacgtagggtattgaatgagagtggagatgggagtagttgg

SEQ ID NO: 14 - DNA, 26 bp, synthetic construct
  source 1..26: mol_type = other DNA
tatcatatgaatcgctgctgggcgct